(19) United States
(12) Patent Application Publication
(10) Pub. No.: US 2004/0090613 A1
(43) Pub. Date: May 13, 2004

(54) Method for measuring the volume of cells or particles

(76) Inventors: Philippe J. Goix, Oakland, CA (US); Paul J. Lingane, Redwood City, CA (US); Kimvan Tran, Union City, CA (US)

Correspondence Address: Aldo J. Test, DORSEY & WHITNEY LLP, Suite 3400, 4 Embarcadero Center, San Francisco, CA, 94111, US

(21) Appl. No.: 10/622,922

(22) Filed: Jul. 17, 2003

Related U.S. Application Data
(60) Provisional application No. 60/396,830, filed on Jul. 17, 2002.

(51) Int. Cl. G01N 33/48; G01B 11/22

(52) U.S. Cl. 356/39; 356/627

(57) ABSTRACT

A method of determining the volume of cells or particles is described. The method relies upon the displacement of the liquid in a liquid suspension of cells or particles. The liquid suspension is strongly fluorescent and the volume of the cells or particles is measured by measuring the reduction of fluorescent light due to the presence of cells or particles.

### RELATED APPLICATIONS

This application claims priority to provisional application Serial No. 60/396,830 filed Jul. 17, 2002.

### BRIEF DESCRIPTION OF THE INVENTION

This invention relates generally to a method of optically determining the volume of cells or particles and more particularly to a method in which the particles or cells are suspended in a liquid medium and their volume is measured by measuring the volume of liquid that is displaced by cells or particles traveling through a measuring volume.

### BACKGROUND OF THE INVENTION

The analysis of individual cells or particles is important in medical and biochemical research. In many cases, for example in drug research, it is particularly important to monitor the change in the volume of cells in response to being exposed to drug candidates. Individual cells or particles as herein used, includes bacteria, viruses, DNA fragments, cells, molecules and constituents of blood.

### OBJECTS AND SUMMARY OF THE INVENTION

It is a general object of the present invention to provide a simple method of obtaining a measure of cell or particle volume. It will also provide a simple method of monitoring a change in volume or a comparison of relative size in cells or particles

It is a further object of the present invention to provide a method of determining cell or particle volume in which the cells or particles are suspended in liquid medium that contains a strongly fluorescing dye and their volume is determined by measuring the reduction of fluorescent light from an irradiated measurement volume when a particle passes through the volume.

The foregoing and other objects of the invention are achieved by suspending the cells or particles that do not fluoresce or weakly fluoresce in a liquid medium having a strongly fluorescent dye, flowing the suspension past a light beam, which excites a predetermined volume of the liquid so that the dye fluoresces, and then detecting the emitted fluorescent light from said volume to provide an output signal indicative of intensity of the emitted light, whereby when a particle or cell flows through the volume the detected light decreases due to displacement of the liquid medium by the cell or particle and provides a measure of the cell or particle volume.

### BRIEF DESCRIPTION OF THE DRAWINGS

The invention will be more clearly understood from the following description when read in conjunction with the accompanying drawings of which:

FIG. 1 schematically illustrates a suitable apparatus for carrying out the present invention;

FIG. 2 is an enlarged view of the irradiated sample volume;

FIG. 3 illustrates the output signal from the detector of FIG. 1; and

FIG. 4 shows the output signal as a function of particle or cell volume for reference beads and cells being measured.

### DESCRIPTION OF THE PREFERRED EMBODIMENTS

A suitable particle analyzing apparatus for carrying out the present invention is sold by Guava Technologies, Inc., the Guava PCA. Briefly, the apparatus includes a capillary through which a sample with suspended cells or particles is drawn past an impinging light beam that illuminates a predetermined volume of the liquid suspension. The illumination scattered by particles as they flow past the light beam is detected and provides a signal representative of the number of cells or particles. Particles that naturally fluoresce or are tagged to fluoresce emit light at a characteristic wavelength and are detected to provide an output signal. The output signals from the scatter and fluorescence detectors permit an analysis of the particles. The particle analyzer can be configured to obtain a measure of the cell or particle volume in accordance with the present invention.

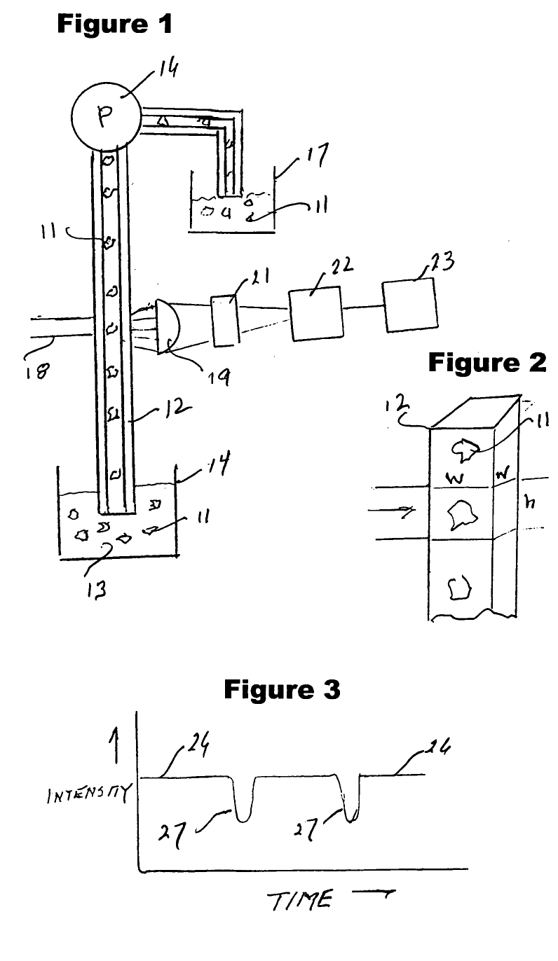

FIG. 1 schematically shows a configuration of apparatus suitable for carrying out the method of the present invention. The suspended particles are drawn through capillary 12 by immersing the end of the capillary in the liquid suspension 13 in the vessel 14. Preferably the capillary is a square capillary. The sample with the suspended particles is aspirated through the capillary by a suitable pump 16 and discharged into the waste container 17. A laser or other suitable light source projects a beam 18 through the capillary. The beam excites the fluid suspension in an analyzing volume defined by the walls of the capillary and the top and bottom edges of the beam. The volume is illustrated in the enlarged view of FIG. 2 and has a volume w·w·h. The size of the capillary is chosen so that the cells or particles are singulated as they travel through the analyzing volume. It is apparent that the analyzing volume can be defined by passages formed in substrates etc. All that is necessary to carry out the invention is to define an analyzing volume through which the particles flow. The fluorescent light emitted by the dye in the analyzing volume is gathered by a lens 19 and passes through a band pass filter 21 that passes light at the fluorescent wavelength and rejects other light. The light is focused onto the photo detector 22 by a lens 19. The output of the photo detector represents the intensity of the light emitted by the fluorescent dye excited by the light beam.

The liquid medium in which the cell or particles are suspended contains a strongly fluorescent dye. The intensity of the emitted light that reaches the detector is proportional to the volume of liquid medium in the light beam. Thus when a cell or particle that does not fluoresce or only fluoresces weakly passes through the analyzing volume it momentarily displaces some of the strongly fluorescent medium. This results in a momentary drop of the total emitted light that reaches the photo detector. The magnitude of the drop can be measured and correlated with the volume of the particle or cell passing through the volume. FIG. 3 schematically illustrates the output signal from the detector. The magnitude of the output signal 26 shows the light without a particle in the analyzing volume while the negative peak 27, which is proportional to the volume of the particle or cell, has a lower intensity. A peak detector 23 receives the output signal from the photo detector and provides an output signal corresponding to the peak amplitude.

In operation, particles spanning a range of known sizes are suspended in the liquid medium and are caused to flow through the analyzing volume. The pulse amplitude is then plotted as a function of particle volume. This provides a reference or calibration curve. Thereafter cells or particles of unknown volume are suspended in a liquid medium and caused to flow through the analyzing volume. The peak amplitude signal is then compared to the reference curve and a measure of the cell volume is obtained.

The following example illustrates use of the present method to provide a particular volume calibration curve and the determination of particle volume. It will be apparent that the calibration curve can be in the form of a look-up table in a process whereby particle volume can be directly provided.

Generally the following steps were performed:

1. Three cell preparations of Sf9 cells were prepared in three different concentrations of PBS to change the osmolality of the cells and expand or shrink the natural cell size.

2. Dyed buffers were prepared to a specific concentration.

3. Four reference samples were prepared using four different sizes of polystyrene beads in the dyed buffer. These were used to create a standard or calibration curve that relates mean detected signal peak height to particle size or volume.

4. Each of the cell preparations were mixed with a dyed buffer and run on the Guava PCA to get mean peak height. The same was done with the four reference bead samples.

5. The mean peak height vs. bead diameter was plotted for the reference beads to determine a best-fit standard curve. This standard curve was used to calculate mean cell size of the cell samples.

The beads were polystyrene bead purchased from Spherotech, Inc. PP-40010 (4 μm nominal bead diameter); PP-60-10 (6 μm nominal bead diameter); PP-100-10 (10 μm nominal bead diameter); and PP-150-10 (15 μm nominal bead diameter). The Sf9 cells were purchased from Gibco Invitrogen Corporation Catalog No. 11496. The 10×PBS was purchased from JRH Biosciences, Catalog No. 59331-79P.

The PBS (phosphate buffer saline) was prepared in three concentrations: 10×PBS, 1×PBS, and 0.2×PBS as follows:

Dilute 10×PBS solution 10 times or 50 times with water to produce 1×PBS or 0.2×PBS, respectively.

The working dye solution was prepared in three concentrations: 10×Dyed PBS, 1×Dyed PBS and 0.2×Dyed PBS as follows:

Approximately 20 mL of stock dye solution (dye molecular weight=581 g/mole) in 1×PBS at 2.4 millimolar concentration was prepared. Then the stock dye was further diluted 20 times in 10×Dyed PBS, 1×Dyed PBS, and 0.2×Dyed PBS, respectively.

1.5 mL each of reference bead samples was prepared by diluting the beads in 1×Dyed PBS as follows:

Reference Sample #1: 4 pm nominal bead diameter 1:10,000 dilution in 1×Dyed PBS;

Reference Sample #2: 6 μm nominal bead diameter 1:1,000 dilution in 1×Dyed PBS;

Reference Sample #3: 10 μm nominal bead diameter 1:400 dilution in 1×Dyed PBS; and

Reference Sample #4: 15 pm nominal bead diameter 1:100 dilution in 1×Dyed PBS.

The bead diameter for each bead suspension was recorded.

Three different preparations of Sf9 cells in 10×, 1×, and 0.2×Dyed PBS were prepared. Each preparation was 1 mL of cells at the concentration of 1 million cells/mL and were labeled “shrunk,” “normal,” and “expanded,” respectively.

The samples were then run. The intensities of the peaks for 1,000 beads each for four reference samples were collected and the mean values for each sample were determined. The three cell samples of each of the three cell preparations 0.2×Dyed PBS only for the “expanded” cells; 1×Dyed PBS only for the “normal” cells; and 10×Dyed PBS only for the “shrunk” cells were run and the intensities of peaks for 1,000 cells each were collected and the mean values were determined.

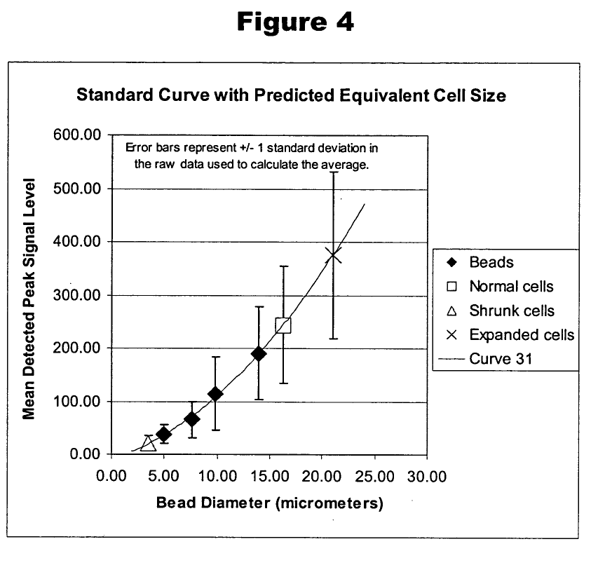

A standard curve 31 using data from the reference samples was plotted in FIG. 4. The cell data was fitted to the standard curve to provide a measure of cell volume. It is clear that this measurement system can distinguish between cell samples of different sizes and provide a measure of cell volumes.

Thus there has been provided a method of determining cell volume in which the volume of the cells is measured by measuring the displacement of a dyed liquid by cells traveling through an analyzing volume.

What is claimed is:

1. The method of obtaining a measure of the volume of cells or particles that comprises the steps of:
suspending the cells or particles in a liquid medium, and
optically measuring the volume of the liquid that is displaced by the cells or particles as they travel through a measuring volume.

2. The method of claim 1 in which the liquid medium is dyed to be strongly fluorescent and in which the predetermined volume is excited by a light source and the reduction in the emitted fluorescent light due to passage of particles in the predetermined volume is measured to provide a measure of the particle volume.

3. The method of determining the volume of cells or particles that comprises the steps of:
forming a suspension of said cells or particles in a liquid medium that is strongly fluorescent,
causing the liquid suspension to flow through a light beam that excites a predetermined volume of the liquid suspension whereby it fluoresces and emits light, and
obtaining measure of the volume of the cells or particles by measuring the reduction of the emitted light caused by the decrease in volume of the fluorescent liquid in the predetermined volume due to the volume of the cells or particles within the predetermined volume.

4. The method of obtaining a measure of the volume of cells or particles that comprises the steps of:
suspending the cells or particles in a liquid medium that is strongly fluorescent,
irradiating a predetermined volume of the liquid medium to cause the liquid to fluoresce and emit light, and
optically determining a measure of the cell volume by measuring the reduction of the emitted light due to cells or particles present in the predetermined volume.

5. The method of obtaining a measure of the volume of cells or particles that comprises the steps of:
preparing a liquid medium with a strongly fluorescent dye,
suspending the cells or particles in the liquid medium,
flowing the suspension through a volume that is defined by a light beam that excites the fluorescent dye and causes it to emit light, and
measuring the reduction of the emitted light when a cell or particle travels through said volume.

6. The method of claim 5 including the additional steps of suspending beads of predetermined volume in the liquid medium and obtaining a measure of the reduction of light for beads of various sizes whereby the reduction of light by cells or particles can be correlated to the data.

7. The method of determining the volume of cells or particles that comprises the steps of:
flowing a liquid medium having a strongly fluorescent dye past a predetermined illuminated volume whereby it emits light or predetermined intensity in the absence of particles or cells of said volume,
detecting the fluorescent light emitted from said volume whereby when a non- or weakly-fluorescent particle or cell is within said volume, the intensity of the fluorescent light decreases, and
correlating the decrease in intensity to the volume of the particles or cells.
